(19) United States
(12) Patent Application Publication
(10) Pub. No.: US 2008/0014244 A1
(43) Pub. Date: Jan. 17, 2008

(54) Implantable medical devices and coatings therefor comprising physically crosslinked block copolymers

(76) Inventors: David C. Gale, San Jose, CA (US); Thierry Glauser, Redwood City, CA (US); Florian Ludwig, Mountain View, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/487,060

(22) Filed: Jul. 13, 2006

(51) Int. Cl. A61K 31/4745; A61F 2/02; C08L 75/06

(52) U.S. Cl. 424/426; 525/440.04; 514/291

(57) ABSTRACT

The current invention relates to physically crosslinked block copolymers, in particular triblock copolymers in which the end blocks are capable of physical crosslinking.

### FIELD

This invention relates to the fields of organic chemistry, polymer chemistry, materials science and medical devices.

### BACKGROUND

Until the mid-1980s, the accepted treatment for atherosclerosis, i.e., narrowing of the coronary artery(ies) was coronary by-pass surgery. While effective and while having evolved to a relatively high degree of safety for such an invasive procedure, by-pass surgery still involves serious potential complications and in the best of cases an extended recovery period.

With the advent of percutaneous tranluminal coronary angioplasty (PTCA) in 1977, the scene changed dramatically. Using catheter techniques originally developed for heart exploration, inflatable balloons were employed to re-open occluded regions in arteries. The procedure was relatively non-invasive, took a very short time compared to by-pass surgery and the recovery time was minimal. However, PTCA brought with it other problems such as vasospasm and elastic recoil of the stretched arterial wall which could undo much of what was accomplished and, in addition, it created a new disease, restenosis, the re-clogging of the treated artery due to neointimal hyperplasia.

The next improvement, advanced in the mid-1980s was use of a stent to hold the vessel walls apart after PTCA. This for all intents and purposes put an end to recoil but did not entirely resolve the issue of restenosis. That is, prior to the introduction of stents, restenosis occurred in from 30-50% of patients undergoing PTCA. Stenting reduced this to about 15-20%, much improved but still more than desirable.

Initially stents were manufactured from metals that were known or found to be relatively safe when implanted in a patient. A great deal of research and development has ensued with the goal of discovering polymers that exhibit all of the beneficial characteristics of the metals but with enhanced safety and chemical and physical properties that permit more tailoring of such devices to their intended end use.

In 2003, drug-eluting stents or DESs were introduced. The drugs initially employed with the DES were cytostatic compounds, compounds that curtailed the proliferation of cells that resulted in restenosis. The occurrence of restenosis was thereby reduced to about 5-7%, a relatively acceptable figure. Today, the DES is the default the industry standard to treatment of atherosclerosis and is rapidly gaining favor for treatment of stenoses of blood vessels other than coronary arteries such as peripheral angioplasty of the femoral artery.

Initially drugs were simply deposited on the surface of stents but, as in the case of stents themselves, polymer chemistry soon began playing, and continue to play a pivotal role in the development of advanced drug delivery systems. Polymers are being developed that permit exquisite control over the release of drugs from DESs from very rapid release to sustained release over periods of time ranging from days, to weeks, even to months and years. To accomplish this, polymers are being designed as drug reservoirs, i.e., matrices in which the drugs are initially dispersed; as expressly rate controlling layers that are coated between the drug reservoir layer and the external environment and as topcoat layers, which may be applied simply to protect the underlying layers or which may double as protective and rate-controlling layers.

One of the key criteria with regard to stents and DESs is the determination of whether the material of which the device is manufactured or with which it is coated will be biostable or biodegradable. If a biostable polymer is selected, i.e., a polymer that does not degrade in a patient's body, its chemical composition is often not of significant concern since it is not intended to break down and enter the patient's system where it might have a deleterious effect. On the other hand, biodegradable polymers are currently preferred for many applications because their ability to decompose in a biological environment confers on them a number of desirable characteristics. For example, the fact that a polymer will biodegrade and can eventually be essentially completely eliminated from a patient's body can avoid the need to invasively remove a DES after its job is done. In addition, by judicious choice of biodegradable polymer, e.g., selecting one that bio-erodes by bulk erosion or one that bio-erodes by surface erosion, the properties of the polymer can be used as an added tool for the fine-tuning of the release rate of a drug.

Of course, if a polymer is going to degrade in a patient's body, it is imperative that it be biocompatible, that is, that its degradation products do no harm to the patient. This requires careful attention to the chemistry of the polymer and the properties of its degradation products. A great deal of work has gone into the effort to find suitable biodegradable polymers.

There remains a need for improved implantable medical devices constructed of or coated with polymers that confer on the device or coating a range of desirable characteristics such as toughness, fracture resistance, shape-stability, flexibility, “tunable” permeability to therapeutic agents, controlled biodegradability, etc. The current invention provides such implantable medical devices.

### SUMMARY

Thus, in one aspect the current invention relates to an implantable medical device comprising:
a device body; and,
a block copolymer having the formula:

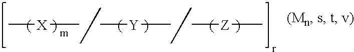

wherein:

m is 0 or 1;

r is an integer from 1 to about 100;

Mₙ is from about 10,000 to about 1,000,000 Da;

s is a number between 0 and 1, inclusive;

t is a number between 0 and 1, inclusive;

v is a number between 0 and 1, inclusive, wherein:

s+t+v=1;

X, if m is not 0, and Z are crystalline or semi-crystalline polymer segments that physically crosslink the block copolymer; and,

Y is an amorphous polymer segment, wherein:
the block copolymer comprises the device body; or,
the block copolymer comprises a layer disposed over at least a portion of the device body; or,
the block copolymer comprises both the device body and a layer disposed over at least a portion of the device body.

In an aspect of this invention, Mₙ is from about 20,000 Da to about 500,000 Da.

In an aspect of this invention, Y is selected from the group consisting of poly(d,l-lactide), poly(meso-lactide), poly(l-lactide-co-trimethylene carbonate) and poly(ethylene glycol).

In an aspect of this invention m is 0.

In an aspect of this invention, when m is 0, Z is biodegradable.

In an aspect of this invention, when m is 0, Z is selected from the group consisting of poly(glycolide), poly(l-lactide), poly(d-lactide), poly(3-hydroxybutyrate), poly(ε-caprolactone) and poly(l,4-dioxan-2-one).

In an aspect of this invention, when m is 0, Z is biostable.

In an aspect of this invention, when m is 0, Z is selected from the group consisting of poly(ethylene terephthalate), poly(butylene terephthalate) and crystallizable hard segments used in polyurethanes.

In an aspect of this invention, when m is 0, Z has a molecular weight less than about 20,000.

In an aspect of this invention, r is 1; and the polymer has the formula:

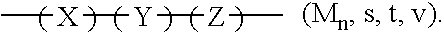

In an aspect of this invention, when r is 1, X and Z are biodegradable.

In an aspect of this invention, when r is 1, X and Z are selected from the group consisting of poly(glycolide), poly(l-lactide), poly(d-lactide), poly(3-hydroxybutyrate), poly(ε-caprolactone) and poly(l,4-dioxan-2one).

In an aspect of this invention, when r is 1, X and Z are biostable.

In an aspect of this invention, when r is 1, X and Z are selected from the group consisting of poly(ethylene terephthalate), poly(butylene terephthalate) and crystallizable hard segments used in polyurethanes.

In an aspect of this invention, when r is 0, X and Z have molecular weights, which are independently less than about 20,000.

In an aspect of this invention, the block copolymer comprises the device body.

In an aspect of this invention, the block copolymer comprises a layer disposed over at least a portion of the device body.

In an aspect of this invention, the layer disposed over at least a portion of the device body comprises one or more of a primer layer, a drug reservoir layer comprising one or more therapeutic agents, a rate-controlling layer and a topcoat layer.

In an aspect of this invention, the layer disposed over at least a portion of the device body comprises at least a drug reservoir layer and the therapeutic agent is selected from the group consisting of rapamycin, 40-O-(2-hydroxyethyl)rapamycin, 40-O-(3-hydroxypropyl)rapamycin, 40-O-(2-hydroxyethyoxy)ethylrapamycin, 40-O-tetrazolylrapamycin, 40-epi(N1-tetrazolyl)rapamycin and clobetasol.

### DETAILED DESCRIPTION

Use of the singular herein includes the plural and visa versa unless expressly stated to be otherwise. That is, unless it is expressly stated otherwise or is obvious from the context, “a” and “the” refer to one or more of whatever the word modifies. For example, “a therapeutic agent” or “the therapeutic agent” may include one such agent, two such agents, etc. Likewise, “a layer” or “the layer” may refer to one, two or more layers and “a polymer” or “the polymer” may mean one polymer or a plurality of polymers. By the same token, words such as, without limitation, “layers” and “polymers” would refer to a plurality of layers or polymers as well as one layer or polymer.

As used herein, any words of approximation such as without limitation, “about,” “essentially,” “substantially” and the like mean that the element so modified need not be exactly what is described but can vary from the description by as much as ±15% without exceeding the scope of this invention.

As used herein, a “polymer segment” refers to a polymeric species that comprises a constitutional unit of a larger polymer. That is, a polymer of this invention has the general formula:

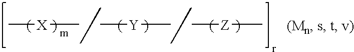

In the above formula, X, Y and Z are the constitutional units of the polymer. A “constitutional unit” simply refers to the, or one of the, repeating units that make up a polymer. For the purposes of this invention, the constitutional units of the polymer are also polymers; thus they are referred to herein as “polymer segments” or sometime simply “segments.” The terms are used interchangeably herein.

In the above formula, “r” refers to the total number of repeats of the X, Y and Z. For the purposes of this invention, r is an integer from 1 to about 100. In a presently preferred embodiment of this invention, m is 1 and r is 1; that is, the polymer is a triblock polymer with one repeat of each of X, Y and Z.

Mₙ represents the number average molecular weight of a polymer of this invention. Again, while any molecular weight that results in a polymer that has the requisite properties to either constitute the body of an implantable medical device or to be disposed as a layer over an implantable medical device is within the scope of this invention, at present the number average molecular weight of a poly(ester-amide) of this invention is from about 10,000 Daltons (Da) to about 1,000,000 Da, preferably at present from about 20,000 Da to about 500,000 Da.

Also in the above formula, s, t and v represent the mole fraction of each of the constitutional units. Each of s, t and v is a number between 0 and 1, inclusive with s+t+v=1. The mole fraction and the size of the constitutional units; that is, the size of the polymer segment are obviously related and it is understood that the designation of one will determine the other. Any combination of mole fractions that affords a polymer that has the properties set forth herein is within the scope of this invention in that those skilled in the art will be readily able to vary such mole fractions and examine the products thereof based on the disclosures herein without undue experimentation.

The polymers of this invention may be regular or random block copolymers. A regular block copolymer has the general structure: . . . x-x-x-y-y-y-z-z-z-x-x-x . . . , while a random block polymer has the general structure: . . . x-x-x-z-z-x-x-y-y-y-y-z-z-z-x-x-z-z-z- . . . . Of course, the juxtaposition of blocks and the total number of blocks in a particular block copolymer of this invention are not in any manner limited by the preceding illustrative generic structures. In the above formula, m is 0 or 1; that is, the polymer may be a triblock copolymer or a diblock copolymer.

While in the above formula, the number of constitutional units may vary and the order of linkage of the units is open, i.e., X may as shown be liked to Y which may be linked to Z, X may as well link directly to Z which then links to another X which links to Y, etc., in a preferred embodiment of this invention, the block copolymer is a triblock copolymer having the formula:

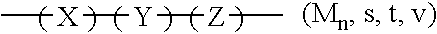

wherein r is 1 so that there is one X, one Y and one z per polymer chain and X is linked to y which is linked to Z. Of course, Mₙ, s, t and v have the same range of values as those set forth for the more generic block copolymer.

As used herein, a “crosslink” refers to a small region in a macromolecule involving at least two discrete polymer chains and from which at least 4 chains emanate. Crosslinking results in the motion of the individual chains involved to be restricted with respect to other chains involved in the crosslink. True crosslinks comprise covalent or ionic links between the chains and are generally referred to as “chemical crosslinks.” For purposes of this invention, however, “physical crosslinks,” which refer to non-bonded interactions between crystalline regions of individual polymer chains, are used.

When a polymer chain comprises sufficient structural regularity, it may come together with other polymer chains in an aligned configuration and ultimately form crystalline structures. Without being held to any particular theory, polymer crystallization is believed to follow the classical growth pattern of crystalline small molecules. That is, crystallization begins with nucleation, the formation of small crystalline particles around a bit of debris in the sea of liquid polymer. These nuclei grow in a hierarchy of ordered structures, namely into lamellae and, eventually, into crystallites. The crystalline regions of polymers exhibit considerable long-range order when subjected to x-ray diffraction examination. The crystalline regions of polymers are quite robust and will maintain in a crosslinked configuration until the melting point, Tₘ, which is a relatively determinate number, of the crystalline regions is reached at which time the crystal structures “melt” similarly to small molecules crystals and become amorphous. Few polymers are 100% crystalline; rather they tend to have discrete regions of crystalline structures and other regions that are amorphous. Thus, the crystalline polymer segments of this invention are referred to as crystalline or semi-crystalline, the latter term referring to a segment that is predominantly but not necessarily completely crystalline. It is presently preferred that a semi-crystalline polymer segment of this invention be at least about 25%, more preferably at least about 50% and most preferably at present at least about 75% crystalline.

Polyurethanes, which include in their backbones the structure—C(O)NH—resulting usually from the reaction of a dihydroxy compound with a diisocyanate, are often multi-block copolymers consisting of alternating hard and soft segments. Hard segments and soft segments are differentiated primarily by their glass transition temperatures, T_{g}. (T_{g}) is the temperature at which a polymer (or a segment of a polymer) changes mechanical properties from those of a rubber (i.e., elastic) to those of a glass (brittle). Below the T_{g} the polymeric molecules have very little translational freedom, i.e., they are unable to move easily or very far in relation to one another. Rather than moving around to adapt to an applied stress, they tend to separate violently so that the polymer breaks or shatters similarly to a pane of glass that is stressed. Above T_{g}, relatively facile segmental motion becomes possible and the polymer chains are able to move around and slip by one another such that when a stress is applied to the polymer it bends and flexes rather than breaks.

While “hard segment” generally refers simply to polymer segments that at a given temperature are below their T_{g}, a special subset of hard segments is those that are crystallizable or in fact crystalline at that same temperature. For the purposes of this invention, a crystallizable hard segment of a polyurethane may be employed as an X, Z, or both, constitutional unit(s) of a block copolymer of this invention so long as the melting point of the crystallizable segment of the polyurethane is above the body temperature of the intended patient. That is, while various mammals have different normal body temperatures, which may change (increase) in the vicinity where an implantable medical device of this invention might be place due to a diseased condition at that locale, the presently preferred patient is a human being, which has a normal body temperature of about 37° C. Thus, the crystallizable hard segment of a polyurethane which can be used to prepare a block copolymer of this invention must be crystalline at or below about 37° C., preferable at or below about 40° C. and most preferable at or below about 50° C.

Those skilled in the art will be able, based on the disclosures herein, to envision numerous crystalline polymers, segments of which would be useful in the invention herein; all such polymer may comprise a polymer segment herein and are within the scope of this invention.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, scaffolds, artificial heart valves and cerebrospinal fluid shunts. An implantable medical device specifically designed and intended solely for the localized delivery of a therapeutic agent is within the scope of this invention.

As used herein, “device body” refers to an implantable medical in a fully formed utilitarian state with an outer surface to which no coating or layer of material different from that of which the device is manufactured has yet been applied. By “outer surface” is meant any surface however spatially oriented that is in contact with bodily tissue or fluids. A common example of a “device body” is a BMS, i.e., a bare metal stent, which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of. In fact, an embodiment of this invention is a device body comprising a block copolymer herein.

Implantable medical devices made of virtually any biocompatible material, i.e., materials presently known to be useful for the manufacture of implantable medical devices and materials that may be found to be so in the future, may be used with a coating of this invention. For example, without limitation, an implantable medical device useful with this invention may be the aforementioned BMS comprising one or more biocompatible metals or alloys thereof including, but not limited to, cobalt-chromium alloy (ELGILOY, L-605), cobalt-nickel alloy (MP-35N), 316L stainless steel, high nitrogen stainless steel, e.g., BIODUR 108, nickel-titanium alloy (NITINOL), tantalum, platinum, platinum-iridium alloy, gold and combinations thereof.

Implantable medical devices may also be made of polymers that are biocompatible and biostable or biocompatible and biodegradable. In general, biodegradable simply means that a particular polymer is degraded in the body by the action of an incipient biological agent, e.g., without limitation, an enzyme, a microbe or a cellular component. Bioabsorbable or bioresorbable on the other hand generally refers to the situation wherein the polymer itself or its degradation products are removed from the body by cellular activity such as, without limitation, phagocytosis. Bioerodible refers to both physical processes such as without limitation dissolution and chemical processes such as, without limitation, backbone cleavage by hydrolysis of the bonds linking constitutional units of a polymer together. As used herein, biodegradable includes bioerodible and bioabsorbable.

As used herein, “biocompatible” refers to a polymer that both in its intact, that is, as synthesized, state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

A “biostable” polymer refers to a polymer that does not significantly biodegrade in a patient's body over an extended period of time, generally in the range of at least many months and preferably many years.

Among useful biocompatible, relatively biostable polymers are, without limitation polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins. Biocompatible, biodegradable polymers include naturally-occurring polymers such as, without limitation, collagen, chitosan, alginate, fibrin fibrinogen, cellulosics, starches, dextran, dextrin, hyaluronic acid, heparin, glycosaminoglycans, polysaccharides and elastin.

One or more synthetic or semi-synthetic biocompatible, biodegradable polymers may also be used to fabricate an implantable medical device useful with this invention. As used herein, a synthetic polymer refers to one that is created wholly in the laboratory while a semi-synthetic polymer refers to a naturally-occurring polymer than has been chemically modified in the laboratory. Examples of synthetic polymers include, without limitation, polyphosphazines, polyphosphoesters, polyphosphoester urethane, polyhydroxyacids, polyhydroxyalkanoates, polyanhydrides, polyesters, polyorthoesters, polyaminoacids, polyoxymethylenes, poly(ester-amides) and polyimides.

Blends and copolymers of the above polymers may also be used and are within the scope of this invention. Based on the disclosures herein, those skilled in the art will recognize those implantable medical devices and those materials from which they may be fabricated that will be useful with the block copolymers of this invention as coatings thereon.

While the above polymers may be used to manufacture implantable medical device bodies separate and apart from those of this invention, i.e., device bodies which for inclusion in this invention would have at least one layer of a block copolymer herein disposed over its surface, many of the above polymers may comprise a polymer segment of a polymer of this invention and as such would be within the scope of this invention.

At present, a preferred implantable medical device of this invention is a stent. A stent refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an artery thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. In fact, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable. In any event, due to the expansion of the stent, any coating thereon must be flexible and capable of elongation.

As use herein, a material that is described as a layer “disposed over” an indicated substrate, e.g., without limitation, a device body or another layer, refers to a relatively thin coating of the material applied, preferably at present, directly to essentially the entire exposed surface of the indicated substrate. By “exposed surface” is meant that surface of the substrate that, in use, would be in contact with bodily tissues or fluids. “Disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, a “primer layer” refers to a coating consisting of a polymer or blend of polymers that exhibit good adhesion characteristics with regard to the material of which the device body is manufactured and good adhesion characteristic with regard to whatever material is to be coated on the device body. Thus, a primer layer serves as an adhesive intermediary layer between a device body and materials to be carried by the device body and is, therefore, applied directly to the device body. Examples, without limitation, of primers include silanes, titanates, zirconates, silicates, parylene, polyacrylates and polymethacrylates, with poly(n-butyl methacrylate) being a presently preferred primer.

As used herein, “drug reservoir layer” refers either to a layer of one or more therapeutic agents applied neat or to a layer of polymer or blend of polymers that has dispersed within its three-dimensional structure one or more therapeutic agents. A polymeric drug reservoir layer is designed such that, by one mechanism or another, e.g., without limitation, by elution or as the result of biodegradation of the polymer, the therapeutic substance is released from the layer into the surrounding environment.

As used herein, “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, the terms “drug” and “therapeutic agent” are used interchangeably.

As used herein, “rate-controlling layer” refers to a polymeric layer that is applied over a drug reservoir layer to modify the rate of release into the environment of the therapeutic agents from the drug reservoir layer. A rate-controlling layer may be used simply to “tune” the rate of release of a therapeutic agent to exactly that desired by the practitioner or it may be a necessary adjunct to the construct because the polymer or blend of polymers with which the therapeutic agent is compatible with regard to coating as a drug reservoir layer may be too permeable to the therapeutic substance resulting in too rapid release and delivery of the therapeutic substance into a patient's body. In such case, a layer may be placed between the drug reservoir layer and the external environment wherein the layer comprises a polymer that, due to its inherent properties or because it has been cross-linked, presents a more difficult to traverse barrier to an eluting drug. The rate-controlling propensity of this layer will depend, without limitation, on such factors as the amount of this polymer in the layer, the thickness of the layer and the degree of cross-linking of the polymer.

As used herein, a “topcoat layer” refers to an outermost layer, that is, a layer that is in contact with the external environment and that is coated over all other layers. The topcoat layer may be applied to provide better hydrophilicity to the device, to better lubricate the device or merely as a device protectant. The topcoat layer, however, may also contain therapeutic agents, in particular if the treatment protocol being employed calls for essentially immediate release of one or more therapeutic agent (these being included in the topcoat layer) followed by the controlled release of another therapeutic agent or agents over a longer period of time. In addition, the topcoat layer may contain one or more “biobeneficial agents.”

A “biobeneficial” agent is one that beneficially affects an implantable medical device by, for example, reducing the tendency of the device to protein foul, increasing the hemocompatibility of the device, and/or enhancing the non-thrombogenic, non-inflammatory, non-cytotoxic, non-hemolytic, etc. characteristics of the device. Some representative biobeneficial materials include, but are not limited to, polyethers such as poly(ethylene glycol)(PEG) and poly(propylene glycol); copoly(ether-esters) such as poly(ethylene oxide-co-lactic acid); polyalkylene oxides such as poly(ethylene oxide) and poly(propylene oxide); polyphosphazenes, phosphoryl choline, choline, polymers and co-polymers of hydroxyl bearing monomers such as hydroxyethyl methacrylate hydroxypropyl methacrylate, hydroxypropylmethacrylamide, poly(ethylene glycol)acrylate, 2-methacryloyloxyethylphosphorylcholine(MPC) and n-vinyl pyrrolidone(VP); carboxylic acid bearing monomers such as methacrylic acid, acrylic acid, alkoxymethacrylate, alkoxyacrylate, and 3-trimethylsilylpropyl methacrylate; polystyrene-PEG, polyisobutylene-PEG, polycaprolactone-PEG (PCL-PEG), PLA-PEG, poly(methyl methacrylate)-PEG(PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functionalized poly(vinyl pyrrolidone); biomolecules such as fibrin, fibrinogen, cellulose, starch, collagen, dextran, dextrin, hyaluronic acid, heparin, glycosamino glycan, polysaccharides, elastin, chitosan, alginate, silicones, PolyActive™, and combinations thereof. PolyActive™ refers to a block copolymer of poly(ethylene glycol) and poly(butylene terephthalate).

An implantable medical device of this invention may include one or more therapeutic agents. Virtually any therapeutic agent found to be useful when incorporated on and implantable medical device may be used in the device and method of this invention. Examples of therapeutic agents include, but are not limited to anti-proliferative, anti-inflammatory, antineoplastic, antiplatelet, anti-coagulant, anti-fibrin, antithrombonic, antimitotic, antibiotic, antiallergic and antioxidant compounds. Thus, the therapeutic agent may be, again without limitation, a synthetic inorganic or organic compound, a protein, a peptide, a polysaccharides and other sugars, a lipid, DNA and RNA nucleic acid sequences, an antisense oligonucleotide, an antibodies, a receptor ligands, an enzyme, an adhesion peptide, a blood clot agent such as streptokinase and tissue plasminogen activator, an antigen, a hormone, a growth factor, a ribozyme, a retroviral vector, an anti-proliferative agent such as rapamycin (sirolimus), 40-O-(2-hydroxyethyl)rapamycin(everolimus), 40-O-(3-hydroxypropyl)rapamycin 40-O-(2-(2-hydroxyethyoxy)ethylrapamycin, 40-O-tetrazolyrapamycin, 40-epi(N1-tetrazolyl)rapamycin(zotarolimus, ABT-578), paclitaxel, docetaxel, methotrexate, azathioprine, vincristine, vinblastine, fluorouracil, doxorubicin hydrochloride, and mitomycin, an antiplatelet compound, an anticoagulant, an antifibrin, an antithrombins such as sodium heparin, a low molecular weight heparin, a heparinoid, hirudin, argatroban, forskolin, vapiprost, prostacyclin, a prostacyclin analogue, dextran, D-phe-pro-arg-chloromethylketone (synthetic antithrombin), dipyridamole, glycoprotein IIb/IIIa platelet membrane receptor antagonist antibody, recombinant hirudin, a thrombin inhibitor such as Angiomax ä, a calcium channel blocker such as nifedipine, colchicine, a fibroblast growth factor (FGF) antagonist, fish oil (omega 3-fatty acid), a histamine antagonist, lovastatin, a monoclonal antibody, nitroprusside, a phosphodiesterase inhibitor, a prostaglandin inhibitor, suramin, a serotonin blocker, a steroid, a thioprotease inhibitor, triazolopyrimidine, a nitric oxide or nitric oxide donor, a super oxide dismutase, a super oxide dismutase mimetic, estradiol, an anticancer agent, a dietary supplement such as vitamins, an anti-inflammatory agent such as aspirin, tacrolimus, dexamethasone and clobetasol, a cytostatic substance such as angiopeptin, an angiotensin converting enzyme inhibitor such as captopril, cilazapril or lisinopril, an antiallergic agent such as permirolast potassium, alpha-interferon, bioactive RGD, and genetically engineered epithelial cells. Other therapeutic agents which are currently available or that may be developed in the future for use with implantable medical devices may likewise be used and all are within the scope of this invention.

Presently preferred therapeutic agents for use with this invention are rapamycin(sirolimus), 40-O-(2-hydroxyethyl)rapamycin(everolimus), 40-O-(3-hydroxypropyl)rapamycin, 40-O-(2-hydroxyethoxy)ethylrapamycyin and 40-O-tetrazole rapamycin(zotarolimus, ABT-578).

What is claimed is:

1. An implantable medical device comprising:
a device body; and,
a block copolymer having the formula: 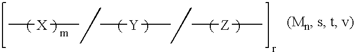
wherein:
m is 0 or 1;
r is an integer from 1 to about 100;
Mₙ is from about 10,000 to about 1,000,000 Da;
s is a number between 0 and 1, inclusive;
t is a number between 0 and 1, inclusive;
v is a number between 0 and 1, inclusive, wherein:
s+t+v=1
X, if m is not 0, and Z are crystalline or semi-crystalline polymer segments that physically crosslink the block copolymer; and,
Y is an amorphous polymer segment, wherein:
the block copolymer comprises the device body; or,
the block copolymer comprises a layer disposed over at least a portion of the device body; or,
the block copolymer comprises both the device body and a layer disposed over at least a portion of the device body.

2. The implantable medical device of claim 1, wherein Mₙ is from about 20,000 Da to about 500,000 Da.

3. The implantable medical device of claim 1, wherein Y is selected from the group consisting of poly(d,l-lactide), poly(meso-lactide), poly(l-lactide-co-trimethylene carbonate and poly(ethylene glycol).

4. The implantable medical device of claim 1, wherein m is 0.

5. The implantable medical device of claim 4, wherein Z is biodegradable.

6. The implantable medical device of claim 5, where Z is selected from the group consisting of poly(glycolide), poly(l-lactide), poly(d-lactide), poly(3-hydroxybutyrate), poly(ε-caprolactone) and poly(l,4-dioxan-2-one).

7. The implantable medical device of claim 4, wherein Z is biostable.

8. The implantable medical device of claim 7, wherein Z is selected from the group consisting of poly(ethylene terephthalate), poly(butylene terephthalate) and crystallizable hard segments used in polyurethanes.

9. The implantable medical device of claim 7, wherein Z has a molecular weight less than about 20,000 Da.

10. The implantable medical device of claim 1, wherein:
r is 1; and
the polymer has the formula:

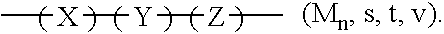

11. The implantable medical device of claim 10, wherein X and Z are biodegradable.

12. The implantable medical device of claim 11, where X and Z are selected from the group consisting of poly(glycolide), poly(l-lactide), poly(d-lactide), poly(3-hydroxybutyrate), poly(ε-caprolactone) and poly(l,4-dioxan-2-one).

13. The implantable medical device of claim 10, wherein X and Z are biostable.

14. The implantable medical device of claim 13, wherein X and Z are selected from the group consisting of poly(ethylene terephthalate), poly(butylene terephthalate) and crystallizable hard segments used in polyurethanes.

15. The implantable medical device of claim 13, wherein X and Z have molecular weights, which are independently less than about 20,000 Da.

16. The implantable medical device of claim 1, wherein the block copolymer comprises the device body.

17. The implantable medical device of claim 1, wherein the block copolymer comprises a layer disposed over at least a portion of the device body.

18. The implantable medical device of claim 17, wherein the layer disposed over at least a portion of the device body comprises one or more of a primer layer, a drug reservoir layer comprising one or more therapeutic agents, a rate-controlling layer and a topcoat layer.

19. The implantable medical device of claim 18, wherein:
the layer disposed over at least a portion of the device body comprises at least a drug reservoir layer; and,
the therapeutic agent is selected from the group consisting of rapamycin, 40-O-(2-hydroxyethyl)rapamycin, 40-O-(3-hydroxypropyl)rapamycin, 40-O-(2-hydroxyethyoxy)ethylrapamycin, 40-O-tetrazolylrapamycin, 40-epi(N1-tetrazolyl)rapamycin and clobetasol.
